(19) United States
(12) Patent Application Publication
(10) Pub. No.: US 2020/0237270 A1
(43) Pub. Date: Jul. 30, 2020

(54) APPARATUS AND METHOD FOR MEASURING BIO-SIGNAL

(71) Applicant: SAMSUNG ELECTRONICS CO., LTD., Suwon-si (KR)

(72) Inventors: Yeol Ho Lee, Anyang-si (KR); Won Jong Jung, Seoul (KR); Kak Namkoong, Seoul (KR); Jung Yong Nam, Hwaseong-si (KR); Joon Hyung Lee, Seongnam-si (KR); Ki Young Chang, Seoul (KR)

(73) Assignee: SAMSUNG ELECTRONICS CO., LTD., Suwon-si (KR)

(21) Appl. No.: 16/731,241

(22) Filed: Dec. 31, 2019

(30) Foreign Application Priority Data
Jan. 30, 2019 (KR) 10-2019-0012236

(51) Int. Cl. A61B 5/145; A61B 5/15

(52) U.S. Cl. CPC A61B 5/14532; A61B 5/150022; A61B 5/14514; A61B 5/14546

(57) ABSTRACT

An apparatus for measuring a bio-signal may include an interstitial fluid extraction assembly configured to extract interstitial fluid from skin of a user, a sensor configured to measure at least one of an impedance and an optical characteristic of the extracted interstitial fluid, and a processor configured to estimate a concentration of an analyte based on at least one of the impedance and the optical characteristic.

### CROSS-REFERENCE TO RELATED APPLICATION(S)

This application claims priority from Korean Patent Application No. 10-2019-0012236, filed on Jan. 30, 2019, in the Korean Intellectual Property Office, the disclosure of which is incorporated herein by reference in its entirety.

### BACKGROUND

### 1. Field

Apparatuses and methods consistent with example embodiments relate to measuring a bio-signal.

### 2. Description of Related Art

Diabetes mellitus is a chronic disease which is difficult to treat and causes various complications. Accordingly, a blood glucose level should be checked regularly to prevent complications. When insulin is administered, blood glucose should be checked in order to prevent hypoglycemia and control the insulin dosage. Generally, measuring blood glucose requires an invasive method such as drawing blood with a finger prick. The method of measuring blood glucose in an invasive manner has high reliability of measurement, but the use of injection may cause pain during blood sampling, inconvenience, and a risk of infection. Accordingly, in particular, a method of predicting blood glucose by extracting an interstitial fluid from skin using reverse iontophoresis, without directly collecting blood, and measuring a glucose concentration from the extracted interstitial fluid is actively being researched.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Example embodiments provide an apparatus and method for measuring a bio-signal.

According to an aspect of an example embodiment, there is provided an apparatus for measuring a bio-signal, the apparatus including an interstitial fluid extraction assembly configured to extract interstitial fluid from skin of a user, a sensor configured to measure at least one of an impedance and an optical characteristic of the extracted interstitial fluid, and a processor configured to estimate a concentration of an analyte based on at least one of the impedance and the optical characteristic.

The interstitial fluid extraction assembly may include a first storage layer configured to extract the interstitial fluid from the skin using reverse iontophoresis and store the extracted interstitial fluid, a second storage layer configured to store interstitial fluid diffused from the first storage layer, and an interference-blocking layer disposed between the first storage layer and the second storage layer and configured to block electrical and optical signals.

The interference-blocking layer may include a channel that allows the interstitial fluid stored in the first storage layer to diffuse into the second storage layer.

The second storage layer may include a vent that permits diffusion of the interstitial fluid from the first storage layer.

The sensor may include at least one of an impedance sensor configured to measure the impedance of the interstitial fluid stored in the second storage layer, and an optical sensor configured to measure the optical characteristic of the interstitial fluid stored in the second storage layer.

The sensor may be an impedance sensor including a plurality of electrodes, and an interval between the plurality of electrodes may be adjustable.

The optical sensor may include a light source configured to emit light toward the interstitial fluid stored in the second storage layer, and a photodetector configured to receive an optical signal reflected by the interstitial fluid stored in the second storage layer.

The interstitial fluid extraction assembly may include a storage layer configured to extract the interstitial fluid from the skin using reverse iontophoresis, and store the extracted interstitial fluid. The sensor may include at least one of an impedance sensor configured to measure the impedance of the interstitial fluid stored in the storage layer, and an optical sensor configured to measure the optical characteristic of the interstitial fluid stored in the storage layer.

The interstitial fluid extraction assembly may include an interference-blocking layer disposed between the storage layer and the skin and configured to block electrical and optical signals.

The analyte may be at least one of glucose, triglyceride, cholesterol, protein, lactate, ethanol, uric acid, and ascorbic acid.

The optical characteristic may be at least one of an absorption characteristic, a reflection characteristic, a transmission characteristic, a semi-transmission characteristic, and a scattering characteristic.

The processor may estimate the concentration of the analyte using at least one of an impedance-concentration relationship model that defines a relationship between an impedance of interstitial fluid and a concentration of an analyte, and an optical characteristic-concentration relationship model that defines a relationship between an optical characteristic of interstitial fluid and a concentration of an analyte.

According to an aspect of another example embodiment, there is provided a method of measuring a bio-signal, the method including extracting interstitial fluid from skin of a user, measuring at least one of an impedance and an optical characteristic of the extracted interstitial fluid, and estimating a concentration of an analyte based on at least one of the impedance and the optical characteristic.

The analyte may include at least one of glucose, triglyceride, cholesterol, protein, lactate, ethanol, uric acid, and ascorbic acid.

The optical characteristic may include at least one of an absorption characteristic, a reflection characteristic, a transmission characteristic, a semi-transmission characteristic, and a scattering characteristic.

The estimating of the concentration of the analyte may include estimating the concentration of the analyte using at least one of an impedance-concentration relationship model that defines a relationship between an impedance of interstitial fluid and a concentration of an analyte and an optical characteristic-concentration relationship model that defines a relationship between an optical characteristic of interstitial fluid and a concentration of an analyte.

Other features and aspects will be apparent from the following detailed description, the drawings, and the claims.

### BRIEF DESCRIPTION OF THE DRAWINGS

The above and/or other aspects will be more apparent by describing certain example embodiments, with reference to the accompanying drawings, in which:

FIG. 1 is a diagram illustrating an apparatus for measuring a bio-signal according to an example embodiment;

FIGS. 2A and 2B are diagrams for describing the interstitial fluid extraction assembly of FIG. 1 according to an example embodiment;

FIGS. 3A and 3B are diagrams for describing the interstitial fluid extraction assembly of FIG. 1 according to another example embodiment;

FIGS. 4A and 4B are diagrams for describing the interstitial fluid extraction assembly of FIG. 1 according to another example embodiment;

FIG. 5 is a diagram illustrating an apparatus for measuring a bio-signal according to another example embodiment;

FIG. 6 is a diagram illustrating a method of measuring a bio-signal according to another example embodiment; and

FIG. 7 is a diagram illustrating a wrist type wearable device according to an example embodiment.

### DETAILED DESCRIPTION

The disclosure is provided to assist the reader in gaining a comprehensive understanding of the methods, apparatuses, and/or systems described herein. Various changes, modifications, and equivalents of the systems, apparatuses and/or methods described herein should be apparent to those of ordinary skill in the art. In the disclosure, a detailed description of known functions and configurations may be omitted so as to not obscure the disclosure.

Throughout the drawings and the detailed description, unless otherwise described, the same reference numerals may refer to the same elements, features, and structures. The relative size and depiction of the elements, features, and structures may be exaggerated for clarity, illustration, and convenience.

As used herein, the singular forms of terms may include the plural forms of the terms, unless the context clearly indicates otherwise. It should be further understood that terms such as “comprises,” “comprising,” “includes,” “including,” etc., as used in the disclosure, may specify the presence of stated features, numbers, steps, operations, elements, components, or combinations thereof, and might not preclude the presence or addition of one or more other features, numbers, steps, operations, elements, components, or combinations thereof.

Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, all of a, b, and c, or any variations of the aforementioned examples.

It should also be understood that the elements or components in the disclosure may be discriminated in accordance with their respective main functions. In other words, two or more elements may be integrated into a single element or a single element may be separated into two or more elements in accordance with subdivided functionality. Additionally, each of the elements in the disclosure may perform a part or an entirety of the function of another element as well as its main function, and some of the main functions of each of the elements may be performed exclusively by other elements. Each element may be realized in the form of a hardware component, a software component, and/or a combination thereof.

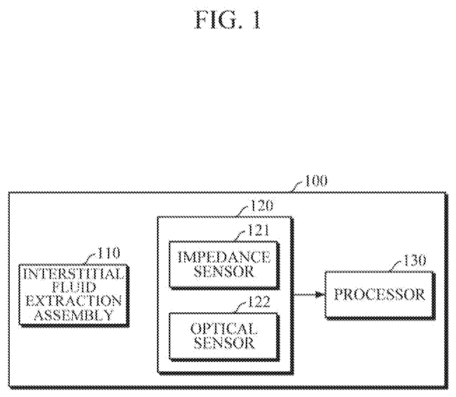

FIG. 1 is a diagram illustrating an apparatus for measuring a bio-signal according to an example embodiment.

A bio-signal measurement apparatus 100 of FIG. 1 may be an apparatus configured to extract interstitial fluid from skin and measure an impedance and/or optical characteristic using the extracted interstitial fluid, and may be mounted in an electronic device or be formed as a separate apparatus surrounding by a housing. The electronic device may include a mobile phone, a smartphone, a tablet computer, a notebook computer, a personal digital assistant (PDA), a portable multimedia player (PMP), a navigation device, an MP3 player, a digital camera, a wearable device, and the like, and the wearable device may include a wrist watch type, a wrist band type, a ring type, a belt type, a necklace type, an ankle band type, a thigh band type, a forearm band type, and the like. However, the electronic device and the wearable device are not limited to the above examples.

Referring to FIG. 1, the apparatus 100 for measuring a bio-signal according to an embodiment may include an interstitial fluid extraction assembly 110, a sensor 120, and a processor 130.

The interstitial fluid extraction assembly 110 may extract interstitial fluid from skin of a user. According to an embodiment, the interstitial fluid extraction assembly 110 may extract interstitial fluid from skin using reverse iontophoresis.

The sensor 120 may measure an impedance and/or optical characteristics of the extracted interstitial fluid. The optical characteristic may include an absorption characteristic, a reflection characteristic, a transmission characteristic, a semi-transmission characteristic, a scattering characteristic, and the like. The sensor 120 may include an impedance sensor 121 and/or an optical sensor 122.

The impedance sensor 121 may measure an impedance of the interstitial fluid by applying a predetermined magnitude of current to the extracted interstitial fluid. The impedance sensor 121 may include a plurality of electrodes, an electric current source configured to apply current to the interstitial fluid via two electrodes among the plurality of electrodes, and a voltmeter configured to measure a voltage applied between two electrodes among the plurality of electrodes. According to an embodiment, the plurality of electrodes of the impedance sensor 121 may be arranged such that an interval between the electrodes is fixed or the interval between the electrodes is adjustable.

The optical sensor 122 may measure an optical characteristic of the interstitial fluid by emitting light of a predetermined wavelength toward the extracted interstitial fluid. The optical sensor 122 may include a light source and a photodetector.

The light source may emit a light ray of a predetermined wavelength, such as, for example, visible light rays, infrared light rays, and the like, toward the interstitial fluid. However, the wavelength of light emitted from the light source may vary according to the purpose of measurement and the type of analyte. In addition, the light source may be configured as a single light emitter, or may be configured in the form of an array of a plurality of light emitters. When the light source is configured as a plurality of light emitters, each light emitter of the plurality of light emitters may emit light of a different wavelength or emit light of the same wavelength. According to an embodiment, the light source may be configured as a light emitting diode (LED), a laser diode, a phosphor, and the like, but is not limited thereto.

The photodetector may receive an optical signal reflected by or scattered from the interstitial fluid. The photodetector may be configured as a single device, or may be configured in the form of an array of a plurality of devices. According to an embodiment, the photodetector may be configured as a photodiode, a phototransistor, or a charge-coupled device (CCD), but is not limited thereto.

The electrodes of the impedance sensor 121 may be variously arranged. For example, the electrodes of the impedance sensor 121 may be spaced at a predetermined distance apart from one another and symmetrically arranged around the optical sensor 122, or may be arranged without a predetermined relation to the optical sensor 122. However, the embodiment is not limited thereto, and the arrangement of the electrodes of the impedance sensor 121 may be variously changed. In addition, the number and arrangement of the light sources and the photodetectors may vary according to the purpose of measurement and the size and form of the electronic device in which the apparatus 100 for measuring a bio-signal is mounted.

The processor 130 may control an overall operation of the apparatus 100 for measuring a bio-signal and may be configured as one or more processors, a memory, or a combination thereof.

The processor 130 may extract interstitial fluid from skin by controlling the interstitial fluid extraction assembly 110 and measure the impedance and/or optical characteristic of the extracted interstitial fluid by controlling the sensor 120.

The processor 130 may estimate a concentration of an analyte by analyzing the measured impedance and/or optical characteristic of the interstitial fluid. The analyte may include glucose, triglyceride, cholesterol, protein, lactate, ethanol, uric acid, ascorbic acid, and the like. If the analyte is glucose, then the concentration of the analyte may represent a blood sugar level.

According to an embodiment, the processor 130 may estimate the concentration of the analyte using at least one of an impedance-concentration relationship model that defines a relationship between an impedance of interstitial fluid and a concentration of an analyte, and an optical characteristic-concentration relationship model that defines a relationship between an optical characteristic of interstitial fluid and a concentration of an analyte.

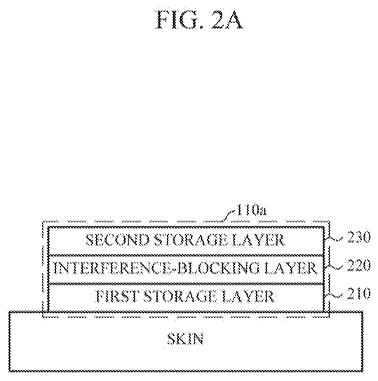

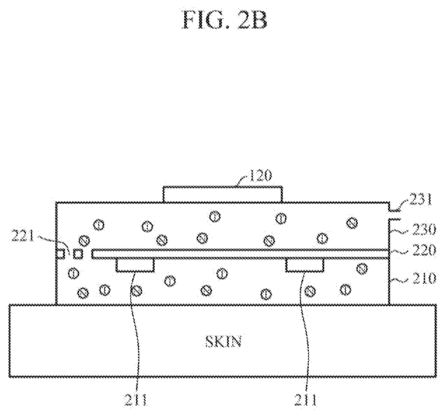

FIGS. 2A and 2B are diagrams for describing the interstitial fluid extraction assembly 110 of FIG. 1 according to an example embodiment.

Referring to FIGS. 2A and 2B, an interstitial fluid extraction assembly 110a may include a first storage layer 210, an interference-blocking layer 220, and a second storage layer 230.

The first storage layer 210 may extract interstitial fluid from skin using reverse iontophoresis and store the extracted interstitial fluid. The first storage layer 210 may include a plurality of electrodes 211 for extracting interstitial fluid from skin using reverse iontophoresis. The first storage layer 210 may be formed of an ion-conductive medium. In this case, the ion-conductive medium may include a conductive polymer gel, a hydrophilic polymer gel, and the like, and may be a medium capable of moving ionic materials when an electric current is applied. According to an embodiment, the first storage layer 210 may be formed of a hydrogel, but is not limited thereto.

The interference-blocking layer 220 may be formed of an electrically insulated material having a light-shielding function and may block electrical and optical signals. The interference-blocking layer 220 may be disposed between the first storage layer 210 and the second storage layer 230. According to an example embodiment, the interference-blocking layer 220 may include a fine channel 221 so that interstitial fluid stored in the first storage layer 210 can be diffused into the second storage layer 230. FIG. 2B illustrates an example in which the fine channel 221 is formed on an edge region of the interference-blocking layer 220, but the embodiment is not limited thereto, and a position at which the fine channel 221 is formed is not particularly limited.

The second storage layer 230 may store the interstitial fluid diffused from the first storage layer 210 through the fine channel 221. The second storage layer 230 may include a vent 231 for promoting the diffusion of the interstitial fluid through the fine channel 221. FIG. 2B illustrates an example in which the vent 231 is formed in a direction that is perpendicular to the direction in which the fine channel 221 is formed, but the embodiment is not limited thereto, and a position at which the vent 231 is formed and a direction in which the vent 231 is disposed are not particularly limited.

The second storage layer 230 may be formed of a transparent material that might not substantially affect light emitted from the sensor 120. In addition, the second storage layer 230 may be formed of an ion-conductive medium (e.g., a hydrogel), but is not limited thereto.

The sensor 120 may be disposed above the second storage layer 230, and may measure an impedance by applying an electric current to the interstitial fluid stored in the second storage layer 230 or measure an optical characteristic by emitting light toward the interstitial fluid stored in the second storage layer 230.

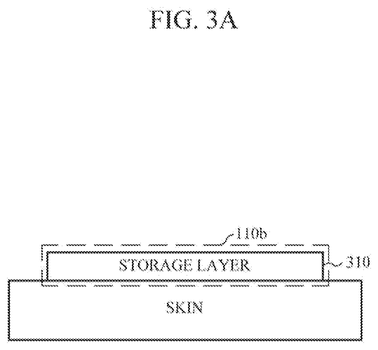

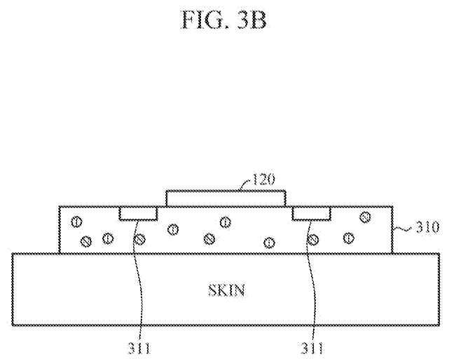

FIGS. 3A and 3B are diagrams for describing the interstitial fluid extraction assembly 110 of FIG. 1 according to an according to another example embodiment.

Referring to FIGS. 3A and 3B, an interstitial fluid extraction assembly 110b may include a storage layer 310.

The storage layer 310 may extract and store interstitial fluid from skin using reverse iontophoresis. The storage layer 310 may include a plurality of electrodes 311 for applying electrical stimuli to the skin. In this case, the plurality of electrodes 311 may be formed as transparent electrodes and the storage layer 310 may be formed of a transparent material in order to reduce an effect on light emitted from the sensor 120. In addition, the storage layer 310 may be formed of an ion-conductive medium (e.g., a hydrogel), but is not limited thereto.

The sensor 120 may be disposed above the storage layer 310 and may measure an impedance by applying an electric current to the interstitial fluid stored in the storage layer 310 or measure an optical characteristic by emitting light toward the interstitial fluid stored in the storage layer 310.

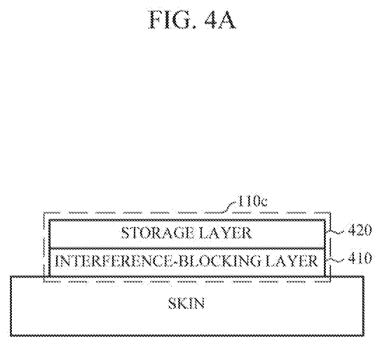

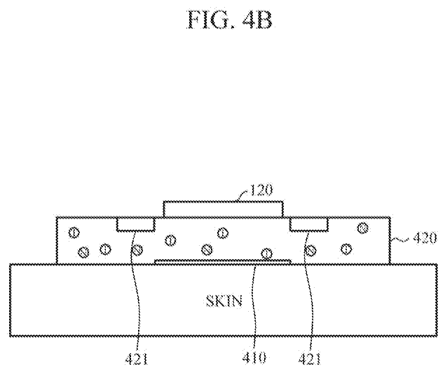

FIGS. 4A and 4B are diagrams for describing the interstitial fluid extraction assembly 110 of FIG. 1 according to an example embodiment.

Referring to FIGS. 4A and 4B, an interstitial extraction assembly 110c may include an interference-blocking layer 410 and a storage layer 420.

The interference-blocking layer 410 may be formed of an electrically insulated material having a light-shielding function and may block electrical and optical signals. The interference-blocking layer 410 may be disposed between the storage layer 420 and skin. According to an example embodiment, the interference-blocking layer 410 may be formed to have a size that permits the interference-blocking layer 410 to block optical signals below the sensor 120, but is not limited thereto. For example, as shown in FIG. 4B, the interference-blocking layer 410 includes a width that is similar to a width of the optical sensor 120, and includes a position that is similar to the optical sensor 120. Further, the interference-blocking layer 410 may include a width that is less than a width of the storage layer 420.

The storage layer 420 may extract and store interstitial fluid from skin using reverse iontophoresis. The storage layer 420 may include a plurality of electrodes 421 for applying electrical stimuli to the skin. The plurality of electrodes 421 may be formed as transparent electrodes and the storage layer 420 may be formed of a transparent material in order to reduce an effect on light emitted from the sensor 120. In addition, the storage layer 420 may be formed of an ion-conductive medium (e.g., a hydrogel), but is not limited thereto.

The sensor 120 may be disposed above the storage layer 420 and may measure an impedance by applying an electric current to the interstitial fluid stored in the storage layer 420 or measure an optical characteristic by emitting light toward the interstitial fluid stored in the storage layer 420.

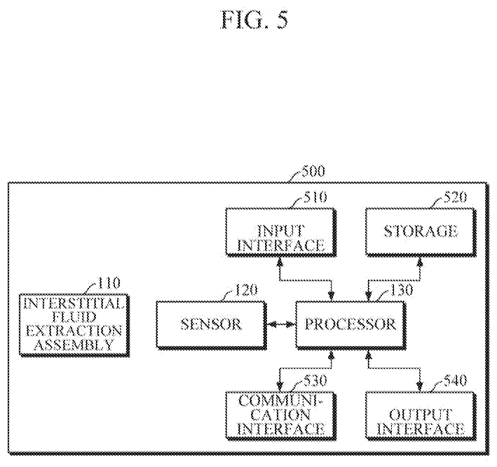

FIG. 5 is a diagram illustrating an apparatus for measuring a bio-signal according to an example embodiment.

A bio-signal measurement apparatus 500 of FIG. 5 may be an apparatus configured to extract interstitial fluid from skin and measure an impedance and/or optical characteristic using the extracted interstitial fluid, and may be mounted in an electronic device or be formed as a separate apparatus surrounding by a housing. The electronic device may include a mobile phone, a smartphone, a tablet computer, a notebook computer, a PDA, a PMP, a navigation device, an MP3 player, a digital camera, a wearable device, and the like, and the wearable device may include a wrist watch type, a wrist band type, a ring type, a belt type, a necklace type, an ankle band type, a thigh band type, a forearm band type, and the like. However, the electronic device and the wearable device are not limited to the above examples.

Referring to FIG. 5, the apparatus 500 for measuring a bio-signal may include an interstitial fluid extraction assembly 110, a sensor 120, a processor 130, an input interface 510, a storage 520, a communication interface 530, and an output interface 540. The interstitial fluid extraction assembly 110, the sensor 120, and the processor 130 may be substantially the same as those described with reference to FIG. 1 to FIG. 4B, and thus detailed descriptions thereof may be omitted.

The input interface 510 may receive various operation signals from a user based on a user input. According to an embodiment, the input interface 510 may include a key pad, a dome switch, a touch pad (e.g., a resistive touch pad, a capacitive touch pad, and the like), a jog wheel, a jog switch, a hardware button, and the like. In particular, the input interface 510 may be a touchpad having a layered structure with a display, and may also be referred to as a touch screen.

A program or instructions for operations of the apparatus 500 for measuring a bio-signal may be stored in the storage 520, and input data, processed data, and output data of the apparatus 500 for measuring a bio-signal may also be stored in the storage 520. In addition, a measured impedance, a measured optical characteristic, and a concentration estimation result of an analyte may be stored in the storage 520. The storage 520 may include a storage medium of at least one type of a flash memory type, a hard disk type, a multimedia card micro type, a card-type memory (e.g., secure digital (SD) or extreme digital (XD) memory), random access memory (RAM), static random access memory (SRAM), read only memory (ROM), electrically erasable programmable read only memory (EEPROM), programmable read only memory (PROM), magnetic memory, magnetic disk, optical disk, and the like. In addition, the apparatus 500 for measuring a bio-signal may communicate with an external storage medium, such as web storage providing a storage function of the storage 520 via the Internet.

The communication interface 530 may communicate with an external device. For example, the communication interface 530 may transmit the input data, stored data, and processed data of the apparatus 500 to the external device, or may receive data associated with estimating a blood concentration of an analyte.

The external device may be medical equipment which uses the input data, stored data, and processed data of the apparatus 500, or may be a printer or a display device for outputting results. In addition, the external device may be a digital TV, a desktop computer, a mobile phone, a smartphone, a tablet computer, a notebook computer, a PDA, a PMP, a navigation device, an MP3 player, a digital camera, a wearable device, or the like, but is not limited thereto.

The communication interface 530 may communicate with the external device using Bluetooth, Bluetooth low energy (BLE), near field communication (NFC), wireless local area network (WLAN) communication, ZigBee communication, infrared data association (IrDA) communication, wireless fidelity (Wi-Fi) direct (WFD) communication, ultra-wideband (UWB) communication, Ant+ communication, Wi-Fi communication, radio-frequency identification (RFID) communication, third generation (3G) communication, fourth generation (4G) communication, fifth generation (5G) communication, and the like. However, these are merely examples, and the embodiment is not limited thereto.

The output interface 540 may output the input data, stored data, and processed data of the apparatus 500. According to an embodiment, the output interface 540 may output the input data, stored data, and processed data of the apparatus 500 via at least one of an audible method, a visual method, and a tactile method. The output interface 540 may include a display, a speaker, a vibrator, and the like.

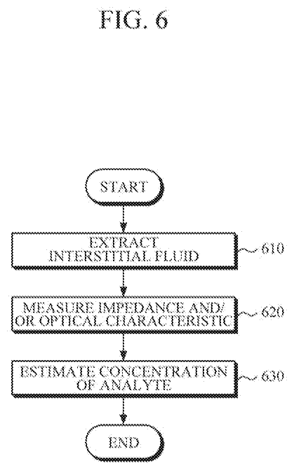

FIG. 6 is a diagram illustrating a method of measuring a bio-signal according to an example embodiment. The method of FIG. 6 may be performed by the apparatuses 100 or 500 of FIG. 1 or 5 to measure a bio-signal.

Referring to FIG. 6, the apparatus for measuring a bio-signal may extract interstitial fluid from skin of a user (S610). According to an embodiment, the apparatus may extract interstitial fluid from skin using reverse iontophoresis.

The apparatus for measuring a bio-signal may measure an impedance and/or optical characteristic of the extracted interstitial fluid (S620). The optical characteristic may include an absorption characteristic, a reflection characteristic, a transmission characteristic, a semi-transmission characteristic, a scattering characteristic, and the like.

The apparatus for measuring a bio-signal may estimate a concentration of an analyte by analyzing the measured impedance and/or optical characteristic of the interstitial fluid (S630). Here, the analyte may include glucose, triglyceride, cholesterol, protein, lactate, ethanol, uric acid, ascorbic acid, and the like. If the analyte is glucose, then the concentration of the analyte may represent a blood sugar level. According to an embodiment, the apparatus may estimate the concentration of an analyte using at least one of an impedance-concentration relationship model that defines a relationship between an impedance of interstitial fluid and a concentration of an analyte and an optical characteristic-concentration relationship model that defines a relationship between an optical characteristic of interstitial fluid and a concentration of an analyte.

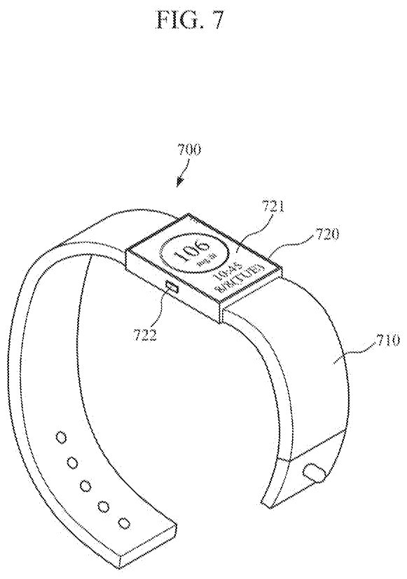

FIG. 7 is a diagram illustrating a wrist type wearable device.

Referring to FIG. 7, the wrist type wearable device 700 may include a strap 710 and a main body 720.

The strap 710 may include two members that are connected to each end of the main body 720 and that are capable of being coupled to each other, or may be integrally formed in the form of a smart band. The strap 710 may be formed of a flexible material to wrap around the wrist of the user such that the main body 720 may be put on the user's wrist.

The main body 720 may have the above-described apparatuses 100 or 500 for measuring a bio-signal mounted therein. In addition, a battery for supplying power to the wrist type wearable device 700 and the apparatuses 100 or 500 for measuring a bio-signal may be embedded in the main body 720.

An interstitial fluid extraction assembly may be disposed in a lower part of the main body 720 so as to be exposed to the wrist of the user. Accordingly, when the user wears the wrist type wearable device 700, the interstitial fluid extraction assembly may contact the user's skin.

The wrist type wearable device 700 may further include a display 721 and an input interface 722 which are disposed on the main body 720. The display 721 may display data processed by the wrist type wearable device 700 and the apparatuses 100 or 500 for measuring a bio-signal, processing result data, and the like. The input interface 722 may receive various operation signals from the user based on a user input.

The embodiments may be implemented as computer readable code stored in a non-transitory computer-readable medium. Code and code segments constituting the computer program may be inferred by a skilled computer programmer in the art. The computer-readable medium includes all types of record media in which computer readable data are stored. Examples of the computer-readable medium include a ROM, a RAM, a CD-ROM, a magnetic tape, a floppy disk, and an optical data storage. Further, the computer-readable medium may be implemented in the form of a carrier wave such as an Internet transmission. In addition, the computer-readable medium may be distributed to computer systems via a network, in which computer-readable code may be stored and executed in a distributed manner.

A number of example embodiments have been described above. However, it should be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. An apparatus for measuring a bio-signal, the apparatus comprising:
an interstitial fluid extraction assembly configured to extract interstitial fluid from skin of a user;
a sensor configured to measure at least one of an impedance and an optical characteristic of the extracted interstitial fluid; and
a processor configured to estimate a concentration of an analyte based on at least one of the impedance and the optical characteristic.

2. The apparatus of claim 1, wherein the interstitial fluid extraction assembly comprises:
a first storage layer configured to extract the interstitial fluid from the skin using reverse iontophoresis and store the extracted interstitial fluid;
a second storage layer configured to store the interstitial fluid diffused from the first storage layer; and
an interference-blocking layer provided between the first storage layer and the second storage layer and configured to block electrical and optical signals.

3. The apparatus of claim 2, wherein the interference-blocking layer comprises a channel that allows the interstitial fluid stored in the first storage layer to diffuse into the second storage layer.

4. The apparatus of claim 2, wherein the second storage layer comprises a vent that permits diffusion of the interstitial fluid from the first storage layer.

5. The apparatus of claim 2, wherein the sensor comprises at least one of an impedance sensor configured to measure the impedance of the interstitial fluid stored in the second storage layer, and an optical sensor configured to measure the optical characteristic of the interstitial fluid stored in the second storage layer.

6. The apparatus of claim 5, wherein the sensor comprises the impedance sensor,
wherein the impedance sensor includes a plurality of electrodes, and
wherein an interval between the plurality of electrodes is adjustable.

7. The apparatus of claim 5, wherein the optical sensor comprises:
a light source configured to emit light toward the interstitial fluid stored in the second storage layer; and
a photodetector configured to receive an optical signal reflected by the interstitial fluid stored in the second storage layer.

8. The apparatus of claim 1, wherein the interstitial fluid extraction assembly comprises a storage layer configured to extract the interstitial fluid from the skin using reverse iontophoresis, and store the extracted interstitial fluid, and
wherein the sensor comprises at least one of an impedance sensor configured to measure the impedance of the interstitial fluid stored in the storage layer, and an optical sensor configured to measure the optical characteristic of the interstitial fluid stored in the storage layer.

9. The apparatus of claim 8, wherein the interstitial fluid extraction assembly further comprises an interference-blocking layer provided between the storage layer and the skin and configured to block electrical and optical signals.

10. The apparatus of claim 1, wherein the analyte includes at least one of glucose, triglyceride, cholesterol, protein, lactate, ethanol, uric acid, and ascorbic acid.

11. The apparatus of claim 1, wherein the optical characteristic includes at least one of an absorption characteristic, a reflection characteristic, a transmission characteristic, a semi-transmission characteristic, and a scattering characteristic.

12. The apparatus of claim 1, wherein the processor is configured to estimate the concentration of the analyte using at least one of an impedance-concentration relationship model that defines a relationship between an impedance of interstitial fluid and a concentration of an analyte, and an optical characteristic-concentration relationship model that defines a relationship between an optical characteristic of interstitial fluid and a concentration of an analyte.

13. A method of measuring a bio-signal, the method comprising:
extracting interstitial fluid from skin of a user;
measuring at least one of an impedance and an optical characteristic of the extracted interstitial fluid; and
estimating a concentration of an analyte based on at least one of the impedance and the optical characteristic.

14. The method of claim 13, wherein the analyte includes at least one of glucose, triglyceride, cholesterol, protein, lactate, ethanol, uric acid, and ascorbic acid.

15. The method of claim 13, wherein the optical characteristic includes at least one of an absorption characteristic, a reflection characteristic, a transmission characteristic, a semi-transmission characteristic, and a scattering characteristic.

16. The method of claim 13, wherein the estimating of the concentration of the analyte comprises estimating the concentration of the analyte using at least one of an impedance-concentration relationship model that defines a relationship between an impedance of interstitial fluid and a concentration of an analyte and an optical characteristic-concentration relationship model that defines a relationship between an optical characteristic of interstitial fluid and a concentration of an analyte.

17. A wearable device configured to be worn on a wrist of a user, the wearable device comprising:
a sensor configured to measure a property of interstitial fluid that is non-invasively collected from skin of the wrist of the user;
a processor configured to estimate a blood glucose level of the user, based on the measured property of the interstitial fluid; and
a display configured to display the estimated blood glucose level of the user.
